(19) United States
(12) Patent Application Publication
(10) Pub. No.: US 2019/0244698 A1
(43) Pub. Date: Aug. 8, 2019

(54) BIOLOGICAL DATA PROCESSING DEVICE, BIOLOGICAL DATA PROCESSING SYSTEM, AND COMPUTER READABLE MEDIUM

(71) Applicants: OMRON HEALTHCARE CO., LTD., Muko-shi (JP); OMRON CORPORATION, Kyoto-shi (JP)

(72) Inventors: Yu MURATA, Kyoto (JP); Toru DENO, Kyoto (JP)

(73) Assignees: OMRON HEALTHCARE CO., LTD., Muko-shi (JP); OMRON CORPORATION, Kyoto-shi (JP)

(21) Appl. No.: 16/385,152

(22) Filed: Apr. 16, 2019

Related U.S. Application Data
(63) Continuation of application No. PCT/JP2017/037713, filed on Oct. 18, 2017.

(30) Foreign Application Priority Data
Oct. 31, 2016 (JP) 2016-213608

(51) Int. Cl. G16H 20/10; G16H 70/40

(52) U.S. Cl. CPC G16H 20/10; G16H 70/40

(57) ABSTRACT

A device is configured to acquire biological data measured from a subject and acquire event data indicating event(s) including taking medicine that has occurred in daily activities of the subject. The device is further configured to, when the event data is acquired, store the acquired event data into a related area for storing data related to the biological data measured before/after an occurrence time of the event. The device is further configured to, when the event data is acquired, determine the kind of the acquired event, and when the event data is acquired and is an event of a predetermined kind, to store the acquired event data into the related area.

The present application is a continuation of International application No. PCT/JP2017/037713, filed Oct. 18, 2017, which claims priority to Japanese Patent Application No. 2016-213608, filed Oct. 31, 2016, the entire contents of each of which are incorporated herein by reference.

### BACKGROUND OF THE INVENTION

### Field of the Invention

This disclosure relates to a device, a system, and a computer readable medium storing program for processing biological data, and more specifically relates to a biological data processing device, a biological data processing system, and a computer readable medium storing a program for processing biological data of a subject together with data of an event in daily activities of the subject.

### Description of the Background Art

For example, the blood pressure of a subject tends to vary according to the situation of daily activities such as taking medicine, meals, and exercise. It is therefore desirable to manage blood pressure values in association with such information of daily activities.

For example, Japanese Patent Laying-Open No. 2006-158879 discloses a configuration to store data of a blood pressure calculated by blood pressure calculation means in a storage unit in association with a measurement condition. Re-publication of PCT International Publication No. WO2010/073692 discloses a configuration to associate a measured value such as blood pressure value and pulse rate with the measurement date and time and lifestyle information (after meals, after exercise, after smoking, at rest). Japanese Patent Laying-Open No. 2012-196508 discloses a configuration to distinguish blood sugar level between before meals and after meals by a before-meal symbol and an after-meal symbol. Japanese Patent Laying-Open No. 2010-213785 discloses a configuration to store a medicine mark, a blood pressure value, and a medicine time in a memory and manage blood pressure measurement and taking medicine (medication) in connection with each other.

### SUMMARY OF THE INVENTION

It is desired to efficiently use a storage area when blood pressure measurement values and the like are stored in association with lifestyle information in a storage unit.

The disclosure is therefore directed to a biological data processing device, a biological data processing system, and a computer readable medium storing a program to enable efficient use of an area for storing biological data.

According to an aspect of this disclosure, a biological data processing device comprises a hardware processor and a storage. The hardware processor is configured to acquire biological data measured from a subject and acquire event data indicating an event that has occurred in daily activities of the subject. The storage is configured to include a data area for storing acquired the biological data and a related area for storing data related to the biological data stored in the data area, and the hardware processor is further configured to, when the event data is acquired, store the acquired event data into the related area for storing data related to the biological data measured before and/or after an occurrence time of the event indicated by the acquired event data. The event includes different kinds of events including taking medicine. The hardware processor is further configured to when the event data is acquired, determine the kind of the acquired event, and when the event data is acquired by the event acquisition unit and is an event of a predetermined kind, store the acquired event data into the related area.

According to another aspect of this disclosure, a biological data processing device comprises a hardware processor and a storage. The hardware processor is configured to acquire biological data measured from a subject and acquire event data indicating an event that has occurred in daily activities of the subject, the event data including an elapsed time since an immediately preceding event occurred. The storage is configured to include a data area for storing the biological data acquired by the hardware processor and a related area for storing data related to the biological data stored in the data area. The hardware processor is further configured to, when the event data is acquired by the hardware processor, store the acquired event data into the related area for storing data related to the biological data measured before and/or after an occurrence time of the event indicated by the acquired event data.

According to another aspect of this disclosure, a biological data processing system comprises a measuring device configured to measure biological data from a subject and a terminal device configured to communicate with the measuring device. The terminal device includes a hardware processor and a storage. The hardware processor is configured to acquire the biological data from the measuring device and acquire event data indicating an event that has occurred in daily activities of the subject. The storage is configured to include a data area for storing the biological data acquired by the hardware processor and a related area for storing data related to the biological data stored in the data area. The hardware processor is further configured to, when the event data is acquired by the event acquisition unit, store the acquired event data into the related area for storing data related to the biological data measured before and/or after an occurrence time of the event indicated by the acquired event data. The event includes different kinds of events including taking medicine. The hardware processor is further configured to, when the event data is acquired, determine the kind of the acquired event, and when the event data is acquired by the hardware processor and is an event of a predetermined kind, store the acquired event data into the related area.

According to another aspect of this disclosure, a biological data processing system comprises a measuring device configured to measure biological data from a subject and a terminal device configured to communicate with the measuring device. The terminal device includes a hardware processor and a storage. The hardware processor is configured to acquire the biological data from the measuring device, and acquire event data indicating an event that has occurred in daily activities of the subject, the event data including an elapsed time since an immediately preceding event occurred. The storage is configured to include a data area for storing the biological data acquired by the hardware processor and a related area for storing data related to the biological data stored in the data area, and the hardware processor is further configured to, when the event data is acquired by the event acquisition unit, store the acquired event data into the related area for storing data related to the biological data measured before and/or after an occurrence time of the event indicated by the acquired event data.

According to another aspect of this disclosure, a computer readable medium stores a program to cause a computer to execute a processing method for biological information. The computer includes a storage being configured to include a data area for storing biological data and a related area for storing data related to the biological data stored in the data area. The processing method comprising the steps of acquiring biological data measured from a subject, acquiring event data indicating an event that has occurred in daily activities of the subject, and when the event data is acquired, storing the acquired event data into the related area for storing data related to the biological data measured before and/or after an occurrence time of the event indicated by the acquired event data. The event includes different kinds of events including taking medicine. The processing method further comprises, when the event data is acquired, determining the kind of the acquired event, and the step of storing includes, when the event data is acquired and is an event of a predetermined kind, storing the acquired event data into the related area.

According to another aspect of this disclosure, a computer readable medium stores a program to cause a computer to execute a processing method for biological information. The computer includes a storage being configured to include a data area for storing biological data and a related area for storing data related to the biological data stored in the data area. The processing method comprises the steps of acquiring biological data measured from a subject, acquiring event data indicating an event that has occurred in daily activities of the subject, the event data including an elapsed time since an immediately preceding event occurred, and when the event data is acquired, storing the acquired event data into the related area for storing data related to the biological data measured before and/or after an occurrence time of the event indicated by the acquired event data.

### BRIEF DESCRIPTION OF THE DRAWINGS

FIG. 1 is a diagram showing an overall configuration of an information processing system 1 according to a first embodiment.

FIG. 2 is a configuration diagram of a sphygmomanometer 21 according to the first embodiment.

FIG. 3 is a configuration diagram of a terminal device 10 according to the first embodiment.

FIG. 4 is a configuration diagram of a server 30 according to the first embodiment.

FIG. 5 is a diagram schematically showing a functional configuration of terminal device 10 according to the first embodiment.

FIG. 6 is a diagram showing the measurement time of biological data 5 and the occurrence time of event data 8 in chronological order according to the first embodiment.

FIG. 7 is a diagram showing an example of data storage in a memory 154 by a storage control unit 124 according to the first embodiment.

FIG. 8 is a flowchart of a process of a measuring device 20 according to the first embodiment.

FIG. 9 is a flowchart of a communication process between the measuring device and the terminal device according to the first embodiment.

FIG. 10 is a diagram showing a display example of a display 158 according to the first embodiment.

FIG. 11 is a diagram showing a configuration of a data management unit 120A of sphygmomanometer 21 according to a second embodiment.

### DESCRIPTION OF THE PREFERRED EMBODIMENTS

Embodiments of the present disclosure will be described in detail with reference to the drawings. In the drawings, the same or corresponding parts are denoted by the same reference signs.

### Overview

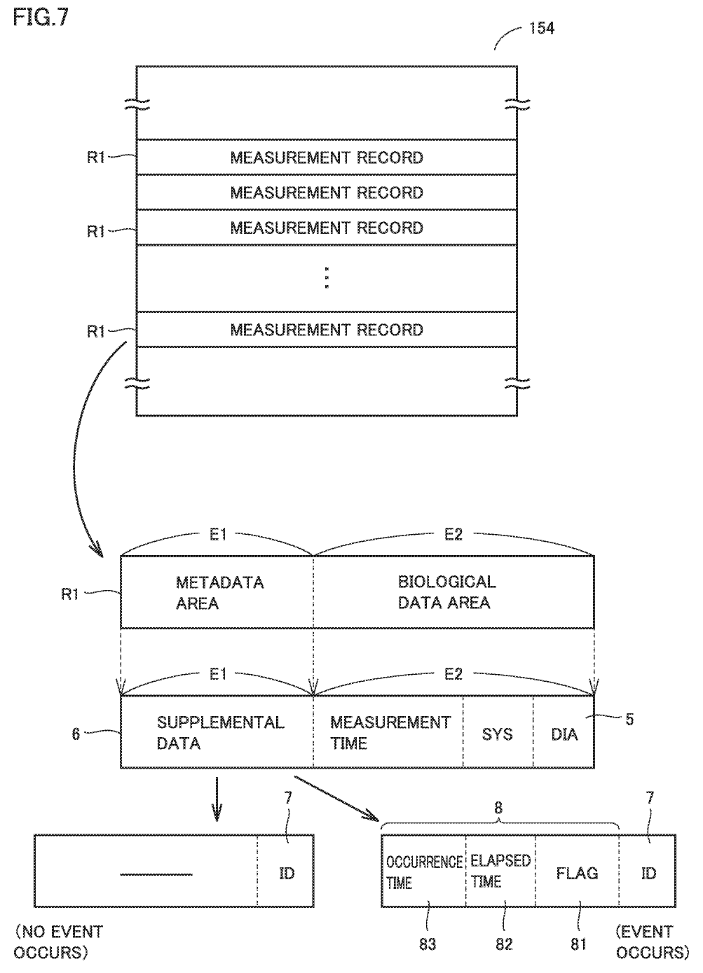

In embodiments, as shown in FIG. 7, event data 8 indicating an event that has occurred before and/or after the time when biological information is measured is stored in a related area (metadata area E1) for storing data related to biological data 5 showing an index of biological information.

With the configuration above, since event data 8 is stored in the related area, it is unnecessary to provide a special area for storing event data 8. Accordingly, the area for storing biological data can be saved.

In embodiments, the biological information measured from a subject may include information indicating a biological state of the subject and information indicating a physical activity (motion) of the subject. The biological data showing an index of the biological information may include blood pressure value, pulse (heart) rate, blood sugar level, the amount of excretion, the amount of perspiration, vital capacity, the amount of sleep, respiratory rate, and body composition values (values indicating body compositions such as body weight, height, muscle mass, bone mass, and the amount of fat).

The amount of activity showing an index of information indicating a physical activity may include “the number of steps” during walking and “calorie consumption” which is energy consumption. The amount of physical activity is not limited to those examples and may include the number of steps up and down, posture, the number of chewing cycles, and the amount of activity of exercise and daily activities (for example, vacuuming, carrying baggage, kitchen work) as a whole. The amount of inactivity may be used as an index of information indicating a physical activity. The amount of inactivity is shown by, for example, the amount of time of sedentary activity such as desk work or TV viewing time.

An “event” that has occurred in daily activities of the subject may include, but not limited to, medication (taking medicine), meal, exercise, smoking, sleep, and excretion.

In embodiments, the measuring device is used by a single user (subject). However, the measuring device may be shared by a plurality of subjects.

### First Embodiment

### System Configuration

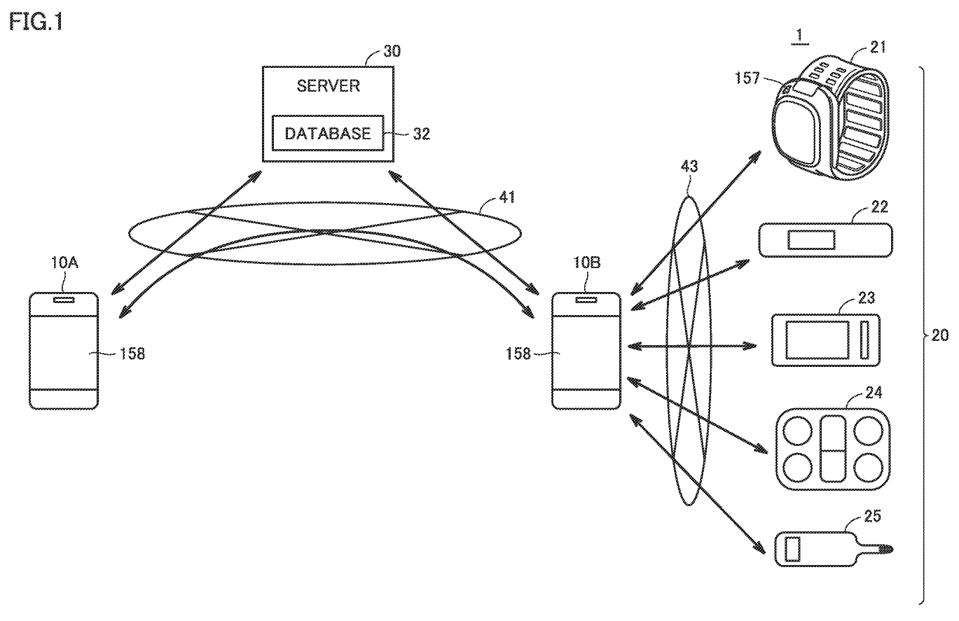

FIG. 1 is a diagram showing an overall configuration of an information processing system 1 according to a first embodiment.

Information processing system 1 is an embodiment of “biological data processing system”. Referring to FIG. 1, information processing system 1 includes terminal devices 10A, 10B used by subjects (also referred to as users), a sphygmomanometer 21, a sleep monitor 22, a pedometer 23, a weight scale and body composition monitor 24, and a thermometer 25, which have the function of measuring the subject's biological information, a server 30, and networks 41, 43. Although terminal device 10A is mainly described below for convenience of explanation, terminal device 10A and terminal device 10B have similar functions. For convenience of explanation, terminal device 10A and terminal device 10B may be collectively referred to as “terminal device 10”.

The biological information measuring device is not limited to sphygmomanometer 21, sleep monitor 22, pedometer 23, weight scale and body composition monitor 24, and thermometer 25 and may be any device for measuring the user's biological information. For example, the biological information measuring device may be an activity tracker. For convenience of explanation, sphygmomanometer 21, sleep monitor 22, pedometer 23, weight scale and body composition monitor 24, and thermometer 25 hereinafter may be collectively referred to as “measuring device 20”.

Here, sphygmomanometer 21 according to the first embodiment is a wristwatch-type sphygmomanometer in which a main unit and a cuff are integrated. Sphygmomanometer 21 may be worn for a long time like a wristwatch to measure pulsation every pulse for consecutive 24 hours or may be always worn to allow the user to push a measurement start button for measurement. Sphygmomanometer 21 thus can always measure the user's blood pressure. As will be described later, sphygmomanometer 21 has a function of measuring the number of steps. In this manner, measuring device 20 may be configured to have the function of measuring different kinds of biological information.

Terminal device 10 is, for example, a smartphone having a touch panel. In the description below, a smartphone is taken as a typical example of “terminal device”. However, the terminal device may be any other terminal device such as a foldable mobile phone, a tablet terminal device, a PC (personal computer), or a PDA (Personal Data Assistance).

Network 41 includes a variety of networks such as the Internet and mobile terminal communication networks for connecting terminal device 10A, terminal device 10B, and server 30 with each other. Network 43 for connecting terminal device 10B with measuring device 20 employs a near-field wireless communication scheme, typically BLE (Bluetooth (registered trademark) low energy). However, network 43 is not limited thereto. A wired communication scheme may be employed, or a wireless LAN (local area network) or any other wireless communication schemes may be employed.

Server 30 includes a database 32 which is an example of the storage area. Server 30 receives data transmitted from each terminal device 10 and stores the received data into database 32. Specifically, server 30 stores the received data into database 32 in association with identification information (terminal ID) of the sender terminal device 10.

Terminal device 10, measuring device 20, and server 30 are embodiments of “data processing device”.

### Configuration of Measuring Device 20

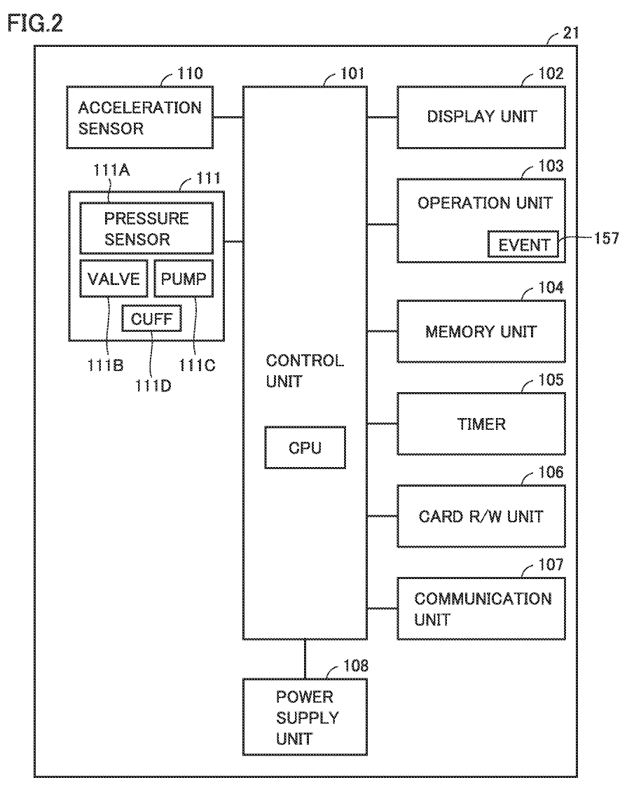

Referring to FIG. 2, a configuration of sphygmomanometer 21 will be described as a typical example of measuring device 20 according to the first embodiment. FIG. 2 is a configuration diagram of sphygmomanometer 21 according to the first embodiment. Referring to FIG. 2, sphygmomanometer 21 includes a control unit 101 including a CPU (Central Processing Unit), a display unit 102 including a display such as a liquid crystal display, an operation unit 103 including keys and buttons for accepting the user's operation to sphygmomanometer 21, a memory unit 104, a timer 105, a card R/W (Read/Write) unit 106 for reading/writing information from/into a variety of recording media such as memory card, a communication unit 107 for communicating with an external information processing device including terminal device 10, and a power supply unit 108.

Memory unit 104 is implemented by, for example, a RAM (Random Access Memory), a ROM (Read-Only Memory), and a flash memory. A program executed by CPU 101 or data used by CPU 101 is stored in a storage area of memory unit 104.

Operation unit 103 includes a button 157 operated by the user when an “event” occurs in the user's daily activities. The operation on button 157 indicates that an “event” has occurred. “Event” may include an event of a kind related to biological information measured by sphygmomanometer 21. More specifically, it may include an event that may be a cause of variation in biological information. Here, for example, the kind of event is “take medicine” but is not limited to “take medicine”.

Sphygmomanometer 21 further includes an acceleration sensor 110 and a blood pressure measuring unit 111 including a circuit for measuring a blood pressure. Acceleration sensor 110 measures an acceleration exerted on sphygmomanometer 21. Blood pressure measuring unit 111 includes a pressure sensor 111A, a valve 111B, a pump 111C, and a cuff 111D wound around a measurement site (arm). During blood pressure measurement, pump 111C supplies air into cuff 111D to expand cuff 111D. The expansion of cuff 111D applies pressure to the measurement site. After applying pressure, valve 111B is opened, whereby air is exhausted from cuff 111D to reduce the pressure on the measurement site. Pressure sensor 111A measures a cuff pressure, which is an internal pressure of cuff 111D in the pressure applying process or the pressure reducing process, and outputs the measured cuff pressure to control unit 101. The cuff pressure during blood pressure measurement corresponds to arterial pressure (information indicating a biological state) measured non-invasively. Control unit 101 calculates a blood pressure value (for example, indices such as systolic blood pressure SYS and diastolic blood pressure DIA), based on the cuff pressure from blood pressure measuring unit 111, for example, according to the oscillometric method. An average blood pressure value may be calculated as the blood pressure value, and a pulse rate may be calculated.

Acceleration sensor 110 measures the acceleration exerted on sphygmomanometer 21 and outputs the measured acceleration to control unit 101. This acceleration corresponds to an acceleration component (information indicating a physical activity) exerted on sphygmomanometer 21 when the user walks. Control unit 101 analyzes the acceleration component from acceleration sensor 110 and calculates the number of steps based on the analysis result. Here, for example, the number of steps for an hour is calculated, and the number of steps for an hour is transmitted to terminal device 10.

The basic configuration of other measuring devices 20 is similar to that of sphygmomanometer 21, except for the function of measuring biological information, and a description thereof is not repeated here.

### Configuration of Terminal Device 10

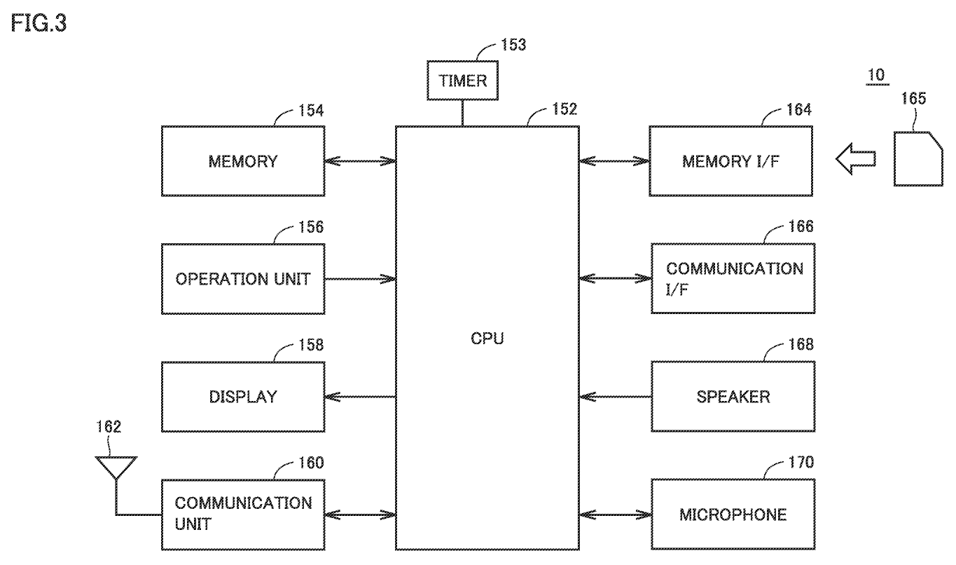

FIG. 3 is a configuration diagram of terminal device 10 according to the first embodiment. Referring to FIG. 3, terminal device 10 includes, as main components, a CPU 152, a timer 153, a memory 154, an operation unit 156 for accepting the user's operation to terminal device 10, a display 158, a communication unit 160 for wireless communication through an antenna 162, a memory interface (I/F) 164, a communication interface (I/F) 166, a speaker 168 for sound output, and a microphone 170 for sound input.

CPU 152 executes a program stored in memory 154 to control each unit.

The storage area of memory 154 is configured with, for example, a RAM (Random Access Memory), a ROM (Read-Only Memory), a flash memory, and a hard disk device. A program executed by CPU 152 or data used by CPU 152 is stored in memory 154.

Operation unit 156 accepts an operation input to terminal device 10. Typically, operation unit 156 is implemented by including a touch panel. The touch panel is provided on display 158. Operation unit 156 may include a switch, a button, and the like.

Wireless communication unit 160 connects to a mobile communication network through antenna 162 and transmits/receives a signal for wireless communication. This enables terminal device 10 to communicate with another communication device (for example, server device 30, another terminal device 10), for example, through a mobile communication network such as LTE (Long Term Evolution).

Memory interface 164 reads out data from an external storage medium 165. CPU 152 reads out data stored in storage medium 165 through memory interface 164 and stores the data into memory 154. CPU 152 reads out data from memory 154 and stores the data into external storage medium 165 through memory interface 164.

Storage medium 165 includes a medium for storing a program or data in a nonvolatile manner, such as a CD (Compact Disc), a DVD (Digital Versatile Disk), a BD (Blu-ray (registered trademark) Disc), a USB (Universal Serial Bus) memory, and an SD (Secure Digital) memory card.

Communication interface (I/F) 166 is a communication interface for exchanging a variety of data between terminal device 10 and measuring device 20 and is implemented by an adaptor or a connector. In the present embodiment, BLE (Bluetooth (registered trademark) low energy) is employed as a communication scheme. However, the communication scheme may be, for example, a wireless communication scheme via a wireless LAN or may be a wired communication scheme using a USB (Universal Serial Bus).

### Configuration of Server

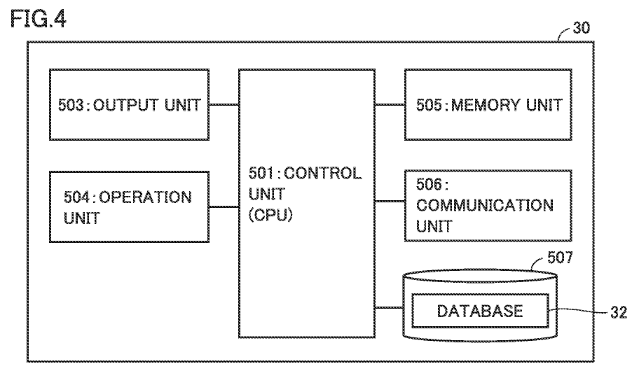

FIG. 4 is a configuration diagram of server 30 according to the first embodiment. Referring to FIG. 4, server 30 includes a control unit 501 including a CPU, an output unit 503 for outputting information, and an operation unit 504 including buttons and switches for accepting a user operation to server 30. Server 30 further includes a communication unit 506 for communicating with terminal device 10, etc. through network 41, a memory unit 505 including a ROM and a RAM storing a program and data, and an HDD (Hard Disc Drive) 507 such as a database 32 for storing a variety of data.

### Transmission of Measurement Data

Control unit 101 of sphygmomanometer 21 generates biological data 5 of blood pressure measurement or the number of steps and transmits the generated biological data 5 to terminal device 10 through communication unit 107. Control unit 101 generates event data 8 when an event occurs and transmits the generated event data 8 to terminal device 10 through communication unit 107. Biological data 5 includes a blood pressure value or the number of steps and a measurement time (day/time/minute). Event data 8 also includes an event occurrence flag and an event occurrence time (day/time/minute). The event occurrence flag may indicate the kind of event (for example, taking medicine). The event occurrence time indicates the time when button 157 is operated.

In the first embodiment, sphygmomanometer 21 transmits biological data 5 to terminal device 10 every time a blood pressure is measured or the number of steps is counted, or transmits event data 8 to terminal device 10 every time an event occurs. Sphygmomanometer 21 may periodically transmit biological data 5 or event data 8. In the first embodiment, sphygmomanometer 21 transmits biological data 5 and event data 8 to terminal device 10 in the form of packets. However, biological data 5 and event data 8 may be transmitted in the form of frames.

### Functional Configuration of Terminal Device 10

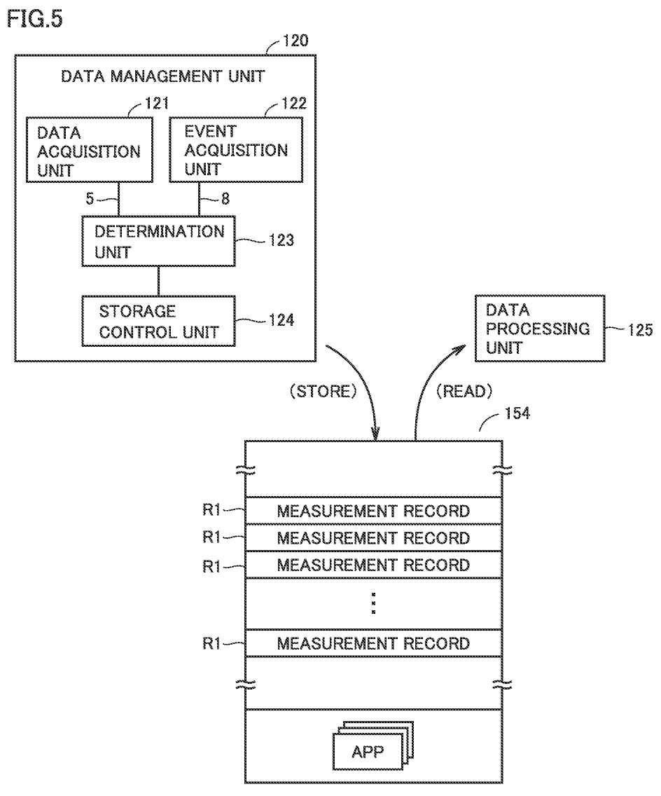

FIG. 5 is a diagram schematically showing a functional configuration of terminal device 10 according to the first embodiment. The functions in FIG. 5 are implemented mainly by a program executed by CPU 152 in control unit 101 or a combination of a program and a circuit. CPU 152 includes a data management unit 120 configured to manage data using memory 154 and a data processing unit 125 configured to process data in memory 154. Data processing unit 125 is implemented by CP152 executing an application program in memory 154. The application program may be downloaded from an external information processing device (for example, server 30) to memory 154.

Data management unit 120 includes a data acquisition unit 121, an event acquisition unit 122, a determination unit 123, and a storage control unit 124 configured to control storage of data into memory 154. Data acquisition unit 121 and event acquisition unit 122 acquire biological data 5 and event data 8, respectively, from data received from sphygmomanometer 21 through communication unit 160. For example, data acquisition unit 121 and event acquisition unit 122 extract biological data 5 or event data 8 from a packet received from sphygmomanometer 21, based on the header of the packet.

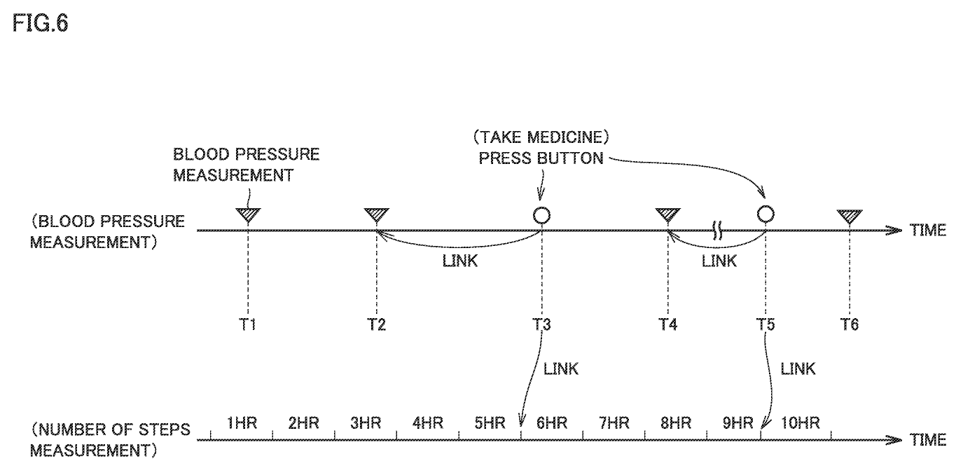

When event acquisition unit 122 acquires event data 8, determination unit 123 determines whether biological information has been measured before and/or after an event occurrence time indicated by event data 8. Storage control unit 124 stores biological data 5 or event data 8 into memory 154, for example, in the form of measurement record R1, based on the result of determination by determination unit 123. The determination process of determination unit 123 will be described later in FIG. 6, and the storing process of storage control unit 124 will be described later in FIG. 6. In the first embodiment, biological data 5 and the corresponding event data 8 are associated in a record format. However, embodiments are not limited to the record format, and biological data 5 and event data 8 may be stored in association with each other in any format.

### Determination Process of Determination Unit 123

Referring to FIG. 6, the determination process of determination unit 123 is described. FIG. 6 is a diagram showing the measurement time of biological data 5 and the occurrence time of event data 8 in chronological order according to the first embodiment. FIG. 6 shows measurement times T1, T2, T4, and T6 of biological data 5 and event occurrence times T3 and T5 of event data 8 on the time axis showing the elapse of time.

### Determination Process when Biological Data 5 is Blood Pressure Value

Determination unit 123 manages biological data 5 accepted from data acquisition unit 121 in chronological order in accordance with measurement times T1, T2, T4, and T6 of biological data 5. Similarly, event data 8 accepted from event acquisition unit 122 is managed in chronological order in accordance with event occurrence times T3 and T5.

Every time event data 8 from event acquisition unit 122 is accepted, determination unit 123 searches the time-series biological data 5 based on the event occurrence time of the accepted event data 8. Based on the result of search, it is determined whether biological data 5 having a measurement time indicating “before” the event occurrence time has been acquired. More specifically, determination unit 123 determines whether there exists biological data 5 indicating a measurement time in a period from “after” the event occurrence time of the event data 8 accepted immediately before to the event occurrence time of the event data 8 accepted this time, in the time-series biological data 5.

In the case of FIG. 6, for example, when event data 8 with event occurrence time T3 is accepted, determination unit 123 searches the time-series biological data 5 based on time T3. Determination unit 123 determines that biological data 5 having measurement time T2 indicating “before” time T3 has been acquired, based on the result of search. Similarly, when event data 8 with event occurrence time T5 is accepted, determination unit 123 determines that biological data 5 having measurement time T4 “before” time T5 has been acquired.

By contrast, in FIG. 6, if event data 8 with event occurrence time T1 is accepted, determination unit 123 determines that biological data 5 having a measurement time indicating “before” time T1 has not been acquired although the time-series biological data 5 is searched based on time T1.

### Determination Process when Biological Data 5 is Number of Steps Data

In the first embodiment, sphygmomanometer 21 transmits biological data 5 of the number of steps to terminal device 10 every hour. In this case, every time event data 8 is accepted from event acquisition unit 122, determination unit 123 searches the time-series biological data 5, based on the event occurrence time of the accepted event data 8, in the same manner as in the blood pressure value described above. Based on the result of search, it is determined whether biological data 5 having a measurement time indicating “before” the event occurrence time has been acquired.

The lower section of FIG. 6 shows a state in which determination unit 123 manages biological data 5 of the number of steps accepted every hour (1 HR) from data acquisition unit 121, in chronological order. In FIG. 6, when event data 8 with event occurrence time T3 is accepted, determination unit 123 determines that biological data 5 of the number of steps in the fifth period (SHR) indicating “before” time T3 has been acquired. Similarly, when event data 8 with event occurrence time T5 is accepted, it is determined that biological data 5 of the number of steps in the ninth period (9 HR) indicating “before” time T5 has been acquired.

By contrast, in FIG. 6, if event data 8 with event occurrence time T1 is accepted, determination unit 123 determines that biological data 5 having a measurement time indicating “before” time T1 has not been acquired although the time-series biological data 5 is searched based on time T1.

In FIG. 6, determination unit 123 is configured to determine whether there exists biological data 5 measured “before” the event occurrence time of event data 8. However, determination unit 123 may be configured to determine whether there exists biological data 5 measured “after” the event occurrence time of event data 8. Alternatively, determination unit 123 may be configured to determine whether there exists biological data 5 measured both “before” and “after”.

### Storing Process of Storage Control Unit 124

FIG. 7 is a diagram showing an example of data storage in memory 154 by storage control unit 124 according to the first embodiment. Here, for simplification of explanation, an example of storage of biological data 5 of blood pressure value is illustrated. Storage control unit 124 generates measurement record R1 in accordance with the result of determination by determination unit 123 described with reference to FIG. 6 and stores the generated measurement record R1 into memory 154.

Referring to FIG. 7, memory 154 has an area for storing a plurality of measurement records R1. Measurement record R1 includes a metadata area E1 and a biological data area E2 to store measurement data by sphygmomanometer 21. Biological data area E2 is an area for storing biological data 5 acquired by data acquisition unit 121. Metadata area E1 corresponds to a related area for storing data related to biological data 5 stored in biological data area E2. The related data includes supplemental data 6 for supplementing biological data 5. Supplemental data 6 includes an ID 7 for identifying biological data 5 in biological data area E2. ID 7 typically includes the identifier of a subject. However, embodiments are not limited thereto. When each user owns his/her individual sphygmomanometer 21 and terminal device 10, ID 7 may include the identifier of sphygmomanometer 21 and the identifier of terminal device 10.

When determination unit 123 accepts event data 8 and determines that there exists biological data 5 measured “before” the event occurrence time as described in FIG. 6, storage control unit 124 stores biological data 5 measured “before” the event occurrence time into biological data area E2 of measurement record R1 and stores the accepted event data 8 together with ID 7 into metadata area E1.

Event data 8 includes a flag 81 that may indicate, for example, the kind of event, an elapsed time 82 from the immediately preceding event occurrence time, and the occurrence time 83 of the event. Elapsed time 82 can be used to calculate the occurrence time 83 of the current event from the occurrence time 83 of the immediately preceding event and elapsed time 82. Thus, occurrence time 83 of an event can be omitted (need not to be stored) in metadata area E1, and the memory can be saved.

When determination unit 123 determines that there is no biological data 5 measured “before”, storage control unit 124 may store event data 8 into metadata area E1 of measurement record R1 but may not store biological data 5 into biological data area E2. Therefore, event data 8 and ID 7 are stored in metadata area E1 of measurement record R1 and no data is stored in biological data area E2. Alternatively, storage control unit 124 may store event data 8 into metadata area E1 of measurement record R1 of biological data 5 indicating the measurement time proximate to (before and/or after) the event occurrence time.

In this way, in memory 154, event data 8 can be managed by linking event data 8 with biological data 5 measured “before” the event occurrence time.

### Process Flow of Measuring Device

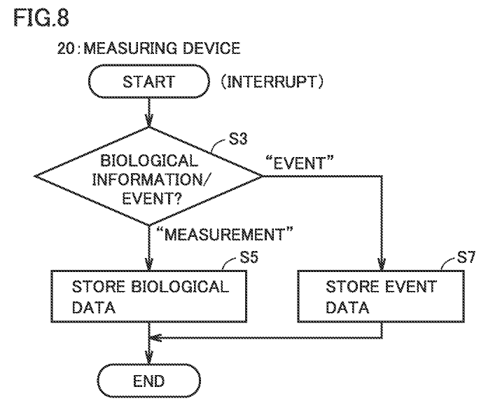

FIG. 8 is a flowchart of a process of measuring device 20 according to the first embodiment. The process flow in FIG. 8 is stored as a program in memory unit 104. The CPU in control unit 101 reads and executes the program to implement the process.

Referring to FIG. 8, the CPU processes an output from blood pressure measuring unit 111, acceleration sensor 110, or operation unit 103 as an interrupt. When the CPU receives biological information from blood pressure measuring unit 111 or acceleration sensor 110 (“measurement” at step S3), the CPU acquires biological data 5 (blood pressure value or the number of steps) based on biological information (cuff pressure or acceleration component) from blood pressure measuring unit 111 or acceleration sensor 110 and stores the acquired biological data 5 (step S5).

When the CPU accepts the operation content of button 157 from an output of operation unit 103 (“event” at step S3), the CPU acquires event data 8 and stores the acquired event data 8 (step S7).

### Communication Process Between Measuring Device and Terminal Device

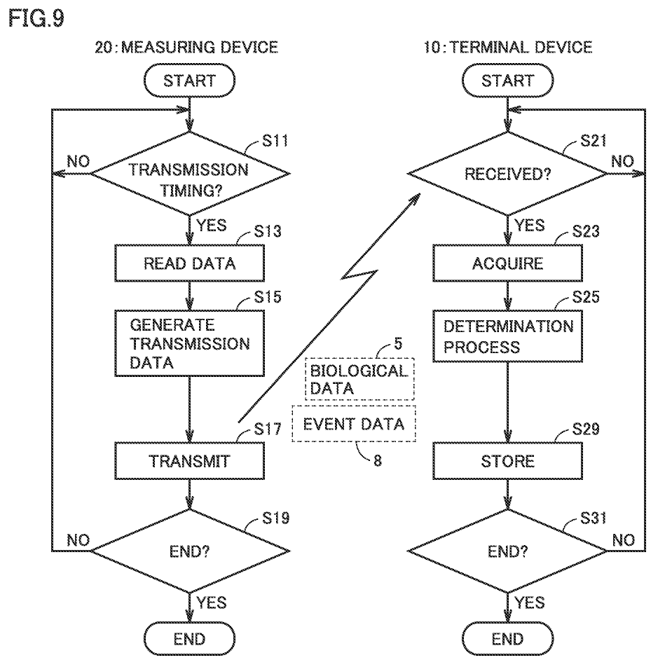

FIG. 9 is a flowchart of a communication process between the measuring device and the terminal device according to the first embodiment. The process flow on the measuring device 20 side in FIG. 9 is stored as a program in memory unit 104. The CPU in control unit 101 reads and executes the program to implement the process. The process flow on the terminal device 10 side is stored as a program in memory 154. CPU 152 reads and executes the program to implement the process. Those processes are performed repeatedly at predetermined time intervals.

Measuring device 20 and terminal device 10 perform a pairing process to enable communication between them. Here, sphygmomanometer 21 is described as measuring device 20.

First, the process of sphygmomanometer 21 is described. Referring to FIG. 9, the CPU of sphygmomanometer 21 determines whether it is the timing to transmit biological data 5 or event data 8 stored in FIG. 8 to terminal device 10 (step S11). In the first embodiment, a transmission timing is determined every time biological information is measured or every time an event occurs (YES at step S11). When the CPU does not determine that it is the transmission timing (NO at step S11), step S11 is repeated.

The CPU reads biological data 5 or event data 8 stored in step S5, S7 (step S13), generates transmission data from the read data (step S15), and transmits the transmission data to terminal device 10 through communication unit 107 (step S17). The CPU determines whether to terminate the process (step S19). For example, the CPU determines to terminate the process (YES at step S19) when sphygmomanometer 21 is powered off. When the CPU does not determine to terminate the process (NO at step S19), the process returns to step S11.

Next, the process of terminal device 10 is described. Referring to FIG. 9, data management unit 120 of terminal device 10 determines whether to receive biological data 5 or event data 8 from sphygmomanometer 21 through communication unit 160 (step S21). When data management unit 120 determines not to receive biological data 5 or event data 8 (NO at step S21), the process at step S21 is repeated.

On the other hand, when data management unit 120 determines that biological data 5 or event data 8 has been received (YES at step S21), data acquisition unit 121 or event acquisition unit 122 acquires biological data 5 or event data 8, respectively, from the data received through communication unit 160 (step S23). Determination unit 123 manages the acquired biological data 5 or event data 8 in chronological order.

When biological data 5 is acquired at step S23, determination unit 123 performs the determination process described in FIG. 6 (step S25). It is then determined whether there exists biological data 5 measured “before” the event occurrence time of the acquired event data 8.

When determination unit 123 determines that there exists biological data 5 measured “before” event data 8, storage control unit 124 stores biological data 5 into biological data area E2 of measurement record R1 and stores event data 8 into the corresponding metadata area E1 (step S29). On the other hand, when determination unit 123 determines that there exists no biological data 5 measured “before” event data 8, storage control unit 124 stores event data 8 into metadata area E1 of measurement record R1 (step S29). Biological data 5 is not stored in biological data area E2 of this measurement record R1. Alternatively, event data 8 may be stored into metadata area E1 of measurement record R1 of biological data 5 indicating the measurement time proximate to (before and/or after) the event occurrence time.

Data management unit 120 determines whether to terminate the process (step S31). For example, when terminal device 10 is powered off, data management unit 120 determines to terminate the process (YES at step S31). When data management unit 120 does not determine to terminate the process (NO at step S31), the process returns to step S21.

Data management unit 120 may encrypt data of measurement record R1 in memory 154. In this case, each application program in data processing unit 125 receives a decryption key from server 30 and reads and decrypts data in memory 154 using the received decryption key.

### Display Example

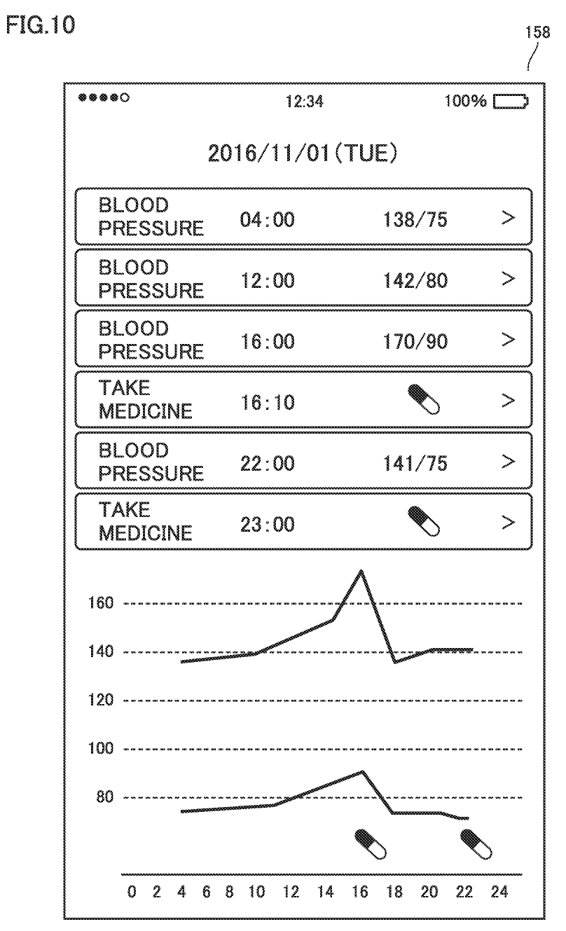

FIG. 10 is a diagram showing a display example of display 158 according to the first embodiment. Referring to FIG. 10, data processing unit 125 displays biological data 5 in biological data area E2 of measurement record R1 on display 158 in association with event data 8 stored in the corresponding metadata area E1.

Specifically, data processing unit 125 reads measurement record R1 in memory 154 and generates display data in accordance with the content of the read measurement record R1. Data processing unit 125 drives display 158 based on the display data. An image corresponding to the display data thus appears on display 158.

In the upper region of the screen on display 158 in FIG. 10, biological data 5 of measurement record R1 and event data 8 linked therewith are displayed in the form of a list in chronological order. In the lower region of the same screen, data is displayed in a mode different from the upper region. Specifically, in the lower region, the blood pressure value (systolic blood pressure SYS, diastolic blood pressure DIA) is shown by a line graph in chronological order, and an event mark (drug capsule mark) is displayed at the timing when an event occurs in association with the line graph.

The image in the upper region and the image in the lower region may be displayed in different screens. The mode of display is not limited to the list form or the line graph as long as biological data 5 and event data 8 can be displayed in association with each other according to the measurement time (event occurrence time).

The screen in FIG. 10 notifies that the blood pressure value after taking medicine is relatively low. The user can confirm that the user takes medicine as prescribed and can check whether the user forgets taking medicine, from the information on the screen.

According to the first embodiment, as shown in FIG. 7, terminal device 10 can store event data 8 and biological data 5 measured “before” the event occurrence time thereof in measurement record R1 and thereby manage them in associated with each other. In addition, since event data 8 is stored in metadata area E1 essentially for storing supplemental data 6, it is unnecessary to prepare a special area for storing event data 8, thereby saving memory 154 of terminal device 10.

### Second Embodiment

A second embodiment is a modification to the first embodiment. In the first embodiment, data management unit 120 of terminal device 10 manages event data 8 in associated with biological data 5. However, in the second embodiment, a data management unit 120A of sphygmomanometer 21 performs this management.

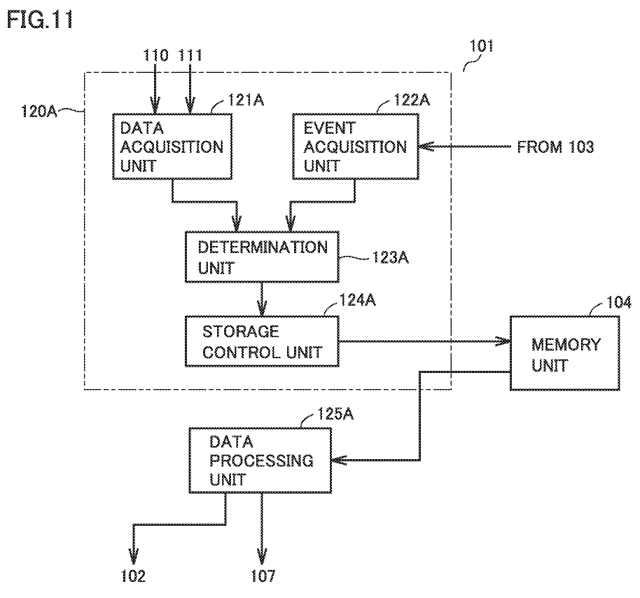

FIG. 11 is a diagram showing a configuration of data management unit 120A of sphygmomanometer 21 according to the second embodiment. Referring to FIG. 11, the CPU of control unit 101 includes a data management unit 120A and a data processing unit 125A. The function of each unit in FIG. 11 is mainly implemented by a program executed by the CPU of control unit 101 or a combination of a program and a circuit. The CPU includes data management unit 120A configured to manage data using memory unit 104 and data processing unit 125A configured to process data in memory unit 104. Data processing unit 125A is implemented by the CPU executing an application program stored in memory unit 104. The application program is stored into memory unit 104 from an external information processing device (for example, server 30) via terminal device 10. Alternatively, the application program is stored into memory unit 104 from an external storage medium via card R/W unit 106.

Data management unit 120A includes a data acquisition unit 121A, an event acquisition unit 122A, a determination unit 123A, and a storage control unit 124A configured to control storage of data into memory unit 104.

Data acquisition unit 121A acquires biological data (blood pressure value or the number of steps) 5 from biological information (cuff pressure or acceleration component) output from sphygmomanometer 21. Event acquisition unit 122A acquires event data 8 when button 157 is operated, in accordance with the operation content from operation unit 103.

When event acquisition unit 122A acquires event data 8, determination unit 123A determines whether there exists biological data 5 measured “before” the event occurrence time indicated by the acquired event data 8, in accordance with biological data 5 managed in chronological order as shown in FIG. 6.

Storage control unit 124A generates measurement record R1 in accordance with the procedure explained in FIG. 7, based on the determination result of determination unit 123A, and stores the generated measurement record R1 into memory unit 104.

Data processing unit 125 reads data of measurement record R1 from memory unit 104 and processes the read biological data. In this case, the read data or the process result is transmitted to terminal device 10 through communication unit 107 or displayed through display unit 102 (see FIG. 10).

Also in the second embodiment, event data 8 is stored in metadata area E1 of measurement record R1 essentially for storing supplemental data 6, in the same manner as in the first embodiment, so that a special area for storing event data 8 becomes unnecessary, thereby saving the memory.

### Third Embodiment

In a third embodiment, a modification to the foregoing embodiments is described. In the foregoing embodiments, the user operates button 157 to designate event occurrence. However, designation of event occurrence is not limited to operation of button 157. In the third embodiment, terminal device 10 displays an icon on display 158.

The user can touch an icon at the time of event occurrence to designate an event occurrence to terminal device 10. Terminal device 10 then generates event data 8 when the icon is touched. Determination unit 123 (123A) links event data 8 generated by touching the icon with biological data 5 received from sphygmomanometer 21. In this way, measurement record R1 similar to that in the embodiments can be generated and stored even by touching an ion on display 158.

### Fourth Embodiment

In a fourth embodiment, a modification to the foregoing embodiments is described. In the foregoing embodiments, the kind of event is “take medicine” only. However, in the fourth embodiment, different kinds of event data 8 are linked with biological data 5. Also in the fourth embodiment, the user operates button 157 to designate occurrence of an event every time an event occurs.

Data management unit 120 (120A) of terminal device 10 manages different kinds of events. For example, when the user takes medicine as prescribed after meal and measures a blood pressure, ‘order data’ of “meal”→“take medicine” is registered in data management unit 120. When event data 8 is acquired, determination unit 123 (123A) determines the kind of the event, based on the ‘order data’ according to chronological order of the event data and stores the determined kind (“meal” or “take medicine”) as event data 8.

For example, when the user makes it a rule to take meal after exercise, and takes medicine as prescribed after meal and then measures a blood pressure, ‘order data’ of “exercise”→“meal”→“take medicine” is registered in data management unit 120 (120A). When event data 8 is acquired, determination unit 123 (123A) determines the kind of the event, based on the ‘order data’ according to chronological order of the event data and stores event data 8 of the determined kind (“exercise” or “meal” or “take medicine”).

Data management unit 120 (120A) can acquire the ‘order data’ from the user operation content accepted through operation unit 156.

### Fifth Embodiment

In a fifth embodiment, in a case where different kinds of events occur, every time data acquisition unit 122 (122A) acquires event data 8, determination unit 123 (123A) determines whether biological data 5 corresponding to the kind of the acquired event data 8 has been acquired, in biological data 5 measured before and/or after the event occurrence time of event data 8.

For this determination, the user operates operation unit 156 (103) to set mapping information between the kind of biological data 5 and the event kind in terminal device 10 (sphygmomanometer 21). The mapping information includes, for example, “blood pressure” as the kind of biological data 5 corresponding to the event kinds “take medicine” and “smoking”. The mapping information also includes “number of steps” as the kind of biological data 5 corresponding to the event kind “meal”.

In the fifth embodiment, when event acquisition unit 122 (122A) acquires event data 8 of “take medicine” or “smoking”, determination unit 123 (123A) determines whether “blood pressure” has been measured as biological data 5, in accordance with the mapping information.

When event acquisition unit 122 (122A) acquires event data 8 of “meal”, determination unit 123 (123A) determines whether “number of steps” has been measured as biological data 5 in accordance with the mapping information.

### Sixth Embodiment

In a sixth embodiment, biological data 5 is managed by linking biological data 5 with event data 8, in database 32 of server 30. In the sixth embodiment, CPU 301 of server 30 has a function similar to data management unit 120 (120A). When biological data 5 or event data 8 is received from sphygmomanometer 21 via terminal device 10, CPU 301 stores the received biological data 5 in associated with each other into database 32 in a similar format as in the foregoing embodiments (see FIG. 7).

Data in database 32 may be browsed by data processing unit 125 of terminal device 10 and displayed on display 158 (see FIG. 10).

### Seventh Embodiment

The method of managing event data 8 in associated with biological data 5 as described above using the flowchart can be provided as a program. A program for implementing this method is stored in memory unit 104 of sphygmomanometer 21 (measuring device 20), memory 154 of terminal device 10, and memory unit 505 of server 30, and the CPU reads the program from the memory and executes instruction codes to implement the process.

In the case of sphygmomanometer 21, this program is downloaded from an external information processing device (for example, terminal device 10) to memory unit 104 through communication unit 107 via a communication circuit. Alternatively, the program is downloaded from an external storage medium to memory unit 104 through card R/W 106.

In the case of terminal device 10, this program is downloaded from an external information processing device (for example, server 30) to memory 154 through communication unit 160 via a communication circuit. Alternatively, the program is loaded from storage medium 165 to memory 154 through memory I/F 164.

In the case of server 30, this program is downloaded from an external information processing device to memory unit 505 through communication unit 506 via a communication circuit.

Terminal device 10, measuring device 20 and server 30 each are not limited to a program executed by processor(s) including the CPU and may include, for example, a circuit (or circuitry) such as application specific integrated circuit (ASIC) or field-programmable gate array (FPGA) or may include a combination of a program and a circuit (or circuitry).

According to an aspect of this disclosure, a biological data processing device includes: a data acquisition unit configured to acquire biological data measured from a subject; an event acquisition unit configured to acquire event data indicating an event that has occurred in daily activities of the subject; a storage unit including a data area for storing the biological data acquired by the data acquisition unit and a related area for storing data related to the biological data stored in the data area; and a storage control unit configured to, when the event data is acquired by the event acquisition unit, store the acquired event data into the related area for storing data related to the biological data measured before and/or after an occurrence time of the event indicated by the acquired event data.

Preferably, the biological data acquired by the data acquisition unit includes biological data measured at predetermined time intervals.

Preferably, the event includes different kinds of events including taking medicine. When the event data is acquired, the biological data processing device determines the kind of the acquired event. When the event data is acquired by the event acquisition unit and is an event of a predetermined kind, the storage control unit stores the acquired event data into the related area.

Preferably, the biological data processing device further includes an operation unit configured to accept a user operation to the biological data processing device. The event acquisition unit detects that the event has occurred based on a content of operation accepted by the operation unit.

Preferably, the biological data processing device further includes a data processing unit configured to process the biological data. The data processing unit displays biological data in the data area in association with the event data stored in the related area of data related to the biological data.

Preferably, the event data includes an elapsed time since an immediately preceding event occurred.

A biological data processing system according to another aspect of this disclosure includes a measuring device configured to measure biological data from a subject and a terminal device configured to communicate with the measuring device. The terminal device includes a data acquisition unit configured to acquire the biological data from the measuring device, an event acquisition unit configured to acquire event data indicating an event that has occurred in daily activities of the subject, a storage unit including a data area for storing the biological data acquired by the data acquisition unit and a related area for storing data related to the biological data stored in the data area, and a storage control unit configured to, when the event data is acquired by the event acquisition unit, store the acquired event data into the related area for storing data related to the biological data measured before and/or after an occurrence time of the event indicated by the acquired event data.

A computer readable medium storing a program according to yet another aspect of this disclosure causes a computer to execute a processing method for biological information. The computer includes a storage unit including a data area for storing biological data and a related area for storing data related to the biological data stored in the data area.

The processing method includes the steps of: acquiring biological data measured from a subject; acquiring event data indicating an event that has occurred in daily activities of the subject; and when the event data is acquired, storing the acquired event data into the related area for storing data related to the biological data measured before and/or after an occurrence time of the event indicated by the acquired event data.

Preferably, the event includes taking medicine.

According to the present disclosure, since event data is stored in the related area, it is unnecessary to provide a special area for storing event data, and the area for storing biological data can be efficiently used.

The embodiments disclosed here should be understood as being illustrative rather than being limitative in all respects. The scope of the present invention is shown not in the foregoing description but in the claims, and it is intended that all modifications that come within the meaning and range of equivalence to the claims are embraced here.

What is claimed is:

1. A biological data processing device comprising:
a hardware processor; and
a storage, the hardware processor being configured to:
acquire biological data measured from a subject; and
acquire event data indicating an event that has occurred in daily activities of the subject,
the storage being configured to include a data area for storing acquired the biological data and a related area for storing data related to the biological data stored in the data area; and
the hardware processor being further configured to, when the event data is acquired, store the acquired event data into the related area for storing data related to the biological data measured before and/or after an occurrence time of the event indicated by the acquired event data, wherein
the event includes different kinds of events including taking medicine,
the hardware processor being further configured to:
when the event data is acquired, determine the kind of the acquired event, and
when the event data is acquired by the event acquisition unit and is an event of a predetermined kind, store the acquired event data into the related area.

2. The biological data processing device according to claim 1, wherein the biological data acquired by the hardware processor includes biological data measured at predetermined time intervals.

3. The biological data processing device according to claim 1, wherein the hardware processor is further configured to accept a user operation to the biological data processing device, and detect that the event has occurred based on a content of operation accepted by from the user.

4. The biological data processing device according to claim 1, wherein the hardware processor is further configured to display biological data in the data area in association with the event data stored in the related area of data related to the biological data.

5. The biological data processing device according to claim 1, wherein the event data includes an elapsed time since an immediately preceding event occurred.

6. A biological data processing device comprising:
a hardware processor; and
a storage,
the hardware processor being configured to:
acquire biological data measured from a subject; and
acquire event data indicating an event that has occurred in daily activities of the subject, the event data including an elapsed time since an immediately preceding event occurred,
the storage being configured to include a data area for storing the biological data acquired by the hardware processor and a related area for storing data related to the biological data stored in the data area; and
the hardware processor being further configured to, when the event data is acquired by the hardware processor, store the acquired event data into the related area for storing data related to the biological data measured before and/or after an occurrence time of the event indicated by the acquired event data.

7. The biological data processing device according to claim 6, wherein the event includes taking medicine.

8. The biological data processing device according to claim 6, wherein the biological data acquired by the hardware processor includes biological data measured at predetermined time intervals.

9. The biological data processing device according to claim 6, wherein
the event includes different kinds of events including taking medicine,
the hardware processor is further configured to, when the event data is acquired, determine the kind of the acquired event, and when the event data is acquired by the hardware processor and is an event of a predetermined kind, store the acquired event data into the related area.

10. The biological data processing device according to claim 6, wherein the hardware processor is further configured to accept a user operation to the biological data processing device, and detect that the event has occurred based on a content of operation accepted from the user.

11. The biological data processing device according to claim 6, wherein the hardware processor is further configured to display biological data in the data area in association with the event data stored in the related area of data related to the biological data.

12. A biological data processing system comprising:
a measuring device configured to measure biological data from a subject; and
a terminal device configured to communicate with the measuring device,
the terminal device including:
a hardware processor; and
a storage,
the hardware processor being configured to:
acquire the biological data from the measuring device; and
acquire event data indicating an event that has occurred in daily activities of the subject,
the storage being configured to include a data area for storing the biological data acquired by the hardware processor and a related area for storing data related to the biological data stored in the data area, and
the hardware processor being further configured to, when the event data is acquired by the event acquisition unit, store the acquired event data into the related area for storing data related to the biological data measured before and/or after an occurrence time of the event indicated by the acquired event data, wherein
the event includes different kinds of events including taking medicine,
the hardware processor being further configured to, when the event data is acquired, determine the kind of the acquired event, and when the event data is acquired by the hardware processor and is an event of a predetermined kind, store the acquired event data into the related area.

13. A biological data processing system comprising:
a measuring device configured to measure biological data from a subject; and
a terminal device configured to communicate with the measuring device,
the terminal device including:
a hardware processor; and
a storage,
the hardware processor being configured to acquire the biological data from the measuring device, and acquire event data indicating an event that has occurred in daily activities of the subject, the event data including an elapsed time since an immediately preceding event occurred,
the storage being configured to include a data area for storing the biological data acquired by the hardware processor and a related area for storing data related to the biological data stored in the data area, and
the hardware processor being further configured to, when the event data is acquired by the event acquisition unit, store the acquired event data into the related area for storing data related to the biological data measured before and/or after an occurrence time of the event indicated by the acquired event data.

14. The biological data processing system according to claim 13, wherein the event includes taking medicine.

15. A computer readable medium storing a program to cause a computer to execute a processing method comprising:
acquiring biological data measured from a subject; and
acquiring event data indicating an event that has occurred in daily activities of the subject,
the computer including a storage being configured to include a data area for storing biological data and a related area for storing data related to the biological data stored in the data area,
when the event data is acquired, storing the acquired event data into the related area for storing data related to the biological data measured before and/or after an occurrence time of the event indicated by the acquired event data, wherein
the event includes different kinds of events including taking medicine, and
when the event data is acquired, determining the kind of the acquired event,
wherein storing the acquired event data includes, when the event data is acquired and is an event of a predetermined kind, storing the acquired event data into the related area.

16. A computer readable medium storing a program to cause a computer to execute a processing method comprising:
acquiring biological data measured from a subject; and
acquiring event data indicating an event that has occurred in daily activities of the subject, the event data including an elapsed time since an immediately preceding event occurred,
the computer including a storage being configured to include a data area for storing biological data and a related area for storing data related to the biological data stored in the data area,
when the event data is acquired, storing the acquired event data into the related area for storing data related to the biological data measured before and/or after an occurrence time of the event indicated by the acquired event data.

17. The computer readable medium according to claim 16, wherein the event includes taking medicine.
